(19) United States
(12) Patent Application Publication
(10) Pub. No.: US 2003/0203874 A1
(43) Pub. Date: Oct. 30, 2003

(54) REVITALIZATION FORMULATION

(76) Inventor: JOHN BOTT-WALTERS, SUFFOLK, ENG (US)

Correspondence Address: FINNEGAN HENDERSON FARABOW, GARRETT & DUNNER, 1300 I STREET N W, WASHINGTON, DC, 20005, US

(21) Appl. No.: 09/555,262

(22) PCT Filed: Nov. 18, 1998
(86) PCT No.: PCT/GB98/03474

(30) Foreign Application Priority Data
Nov. 27, 1997 (GB) 9725061.7

(51) Int. Cl. A61K 31/715; A61K 31/375; A61K 31/198

(52) U.S. Cl. 514/54; 514/561; 514/474

(57) ABSTRACT

This invention relates to a formulation combining the additive effects of branched chain amino acids, maltodextrins, and salts for manufacture in the food industry for the purpose of reducing: 1) Alcohol induced decline in well-being, and/or 2) Appetite for alcohol.

This invention relates to a formulation combining the additive effects of branched chain amino acids, maltodextrins, and salts for manufacture in the food industry for the purpose of reducing:

1. Alcohol induced decline in well-being, and/or

2. Appetite for alcohol.

1. Alcohol Induced Decline in Well-Being and/or Hangover

Currently, there is no effective product available for the prevention and or amelioration of alcohol induced decline in well being/hangover that does not contain analgesics.

Alcohol consumption causes liver damage. The combined taking of alcohol and analgesics exacerbates liver damage, but this and other detrimental effects of this course of treatment may be concealed by analgesia.

The present invention prevents or substantially reduces the short term detrimental effects of excess alcohol consumption when taken during or post alcohol consumption immediately prior to sleep. The invention uniquely applies the branched chain amino acids (BCAAs), maltodextrins (a heterogenous mixture of mainly long chain sugars), and salts acting in concert. When used for the prevention and or amelioration of alcohol induced hangover, unexpectedly the BCAAs (which are taken up by the liver [British Medical Bulletin, 48, No. 3, pp 477-495] and mitigate against fatigue induced serotonin production) are highly effective at solving this problem in combination with the maltodextrins (which act to protect the amino acids during their passage through the stomach) plus the salts (which allow for rapid dispersion in water). This is not obvious: the accepted opinion is that alcohol induced hangover is primarily caused by water balance, hyperglycaemia and irritation of the pia mater by tannins and other cogeners found in alcoholic beverages.

2. Suppression of Appetite for Alcohol

Consumption of the formulation in water periodically, for example three times per day, is efficacious in weaning alcoholics off alcohol. Current treatments focus on the blocking of endorphins, GABA receptors, and/or peer pressure. This is the first non-pharmaceutical, entirely food-arade efficacious solution to this problem.

According to a first aspect the present invention provides an aqueous solution typically 250-300 ml in volume containing:

a) Maltodextrin preferably at 4 to 15 Dextrose Equivalents (DE), more preferably at 8 to 12 DE, at 3 to 10% (w/v), even more preferably at 4 to 6% (w/v), and most preferred at 4.5% (w/v);

b) Maltodextrin preferably at 10 to 75 Dextrose Equivalents (DE), more preferably at 10 to 20 DE, most preferred 13 to 15 DE, at 0.1 to 2% (w/v), but most preferred at 0.15% (w/v);

c) Maltodextrin preferably at 6 Dextrose Equivalents (DE) at 1 to 10% (w/v), but most preferred at 4% (w/v);

d) L-leucine at 0.025 to 5 grammes in total, more preferred 0.025 to 0.75 g in total and most preferred 0.05 g in total;

e) L-isoleucine at 0.001 to 5 grammes in total, more preferred 0.01 to 0.75 g in total and most preferred 0.025 g in total;

f) L-valine at 0.001 to 5 grammes in total, more preferred 0.01 to 0.75 g in total and most preferred 0.025 g in total;

g) Preferably L-ascorbic acid at about 0.1 g in total;

h) Preferably anhydrous citric acid at about 0.4 g;

i) Optionally sodium bicarbonate at about 0.3 g.

In addition any required colourings, preservatives and/or flavourings and/or carbonation agent for taste and/or appeal.

In a second aspect the invention provides an aqueous solution typically 250-300 ml in volume containing:

a) about 6% (w/v) maltodextrins—made up of 50% MD6, 25% MD10 and 25% MD20;

b) about 25 mg each of valine and isoleucine;

c) about 50 mg of leucine;

d) about 60 mg of ascorbic acid; and

e) about 400 mg of citric acid.

In a third aspect the invention provides dry formulations which, when dissolved in about 250-300 ml of liquid such as water, provide the solutions described above.

The dry formulation may optionally contain a sweetening agent, such as Aspartame. The metabolic cleavage of Aspartame when the dissolved formulation is ingested has the effect of releasing in vivo a small amount of methanol which improves the efficacy of the product. A preferred amount of Aspartame is about 50 mg.

A formulation according to the invention would normally be prepared in dry form suitable for consumotion as a water-based drink by combination with the appropriate amount of water. Advantageously, the preparation may contain a substance causing effervescence in contact with water: advantageously the action of effervescence may not only disperse the formulation in the water but also cause a level of solubilisation.

One form of the formulation can be made from dry components by blending, and does not require spray-drying.

It will be apparent to those skilled in the art that the preparation in dry form could be mixed with any other liquid, preferably a water based liquid, for consumption. It is envisaged that the dry preparation could be mixed not only with non-alcoholic liquids but also with alcoholic liquids such as beer.

Additionally, it will be apparent to those skilled in the art that the formulation could be consumed as a ready-prepared liquid formulation. Furthermore, it is envisaged that the formulation could be incorporated in other “carriers”: one example would be the preparation of a confectionery item containing the formulation which could be eaten.

Whilst the foregoing description refers to both alcohol induced decline in well being (hangover) and suppression of appetite for alcohol, formulations according to the present invention may be used in the prevention or amelioration of other symptoms such as, by way of non-limiting example, metabolic dysfunction, stress, PMS etc. In particular the formulation according to the invention may be useful in prevention or amelioration of symptoms associated with serotonin release by regulation, suppression or prevention of sertonin production. It will be apparent to those skilled in the art that the formulation of the present invention could be co-administered with one or more other components having serotonin-controlling activity (eg Prozac) and that the BCCA content of the formulation of the present invention could, in appropriate circumstances, be partially or completely substituted with other pharmacologically or physiologically active analogues such as, for example, Prozac.

Not only is the formulation of the invention intended for use by humans, but also it is intended that it might be advantageously administered to animals, particularly in order to prevent or ameliorate the adverse affects of stress on animals. In particular, it is envisaged that the formulation of the invention could be fed to pre-slaughter animals (eg pigs) in order to reduce or overcome stress-related problems known to those skilled in the art.

The choice of maltodextrin used in the formulation according to the invention is not absolutely critical but, as illustrated previously, a preferred formulation may utilise three maltodextrins (a),(b) and (c) above. It is apparent that the DE values referred to above can overlap: it will be understood by those skilled in the art that a choice of maltodextrin could be made within each of the given value ranges (a) and (b) and that the DE value given in (c) falls within the range defined in (a). However, as a general guideline, it is preferred to have at least one maltodextrin of DE value below about 10 and one maltodextrin of DE value above about 10. The DE value has an effect of the rate of uptake and utilisation of the maltodextrin by the consumer of the formulation.

It will also be apparent to those skilled in the art that some or all of the maltodextrin content of the formulation of the invention could be substituted by different carbohydrate monomers, oligomers, and polymers. However, maltodextrin is preferred because it does not provoke a glycaemic response. Nonetheless, it is envisaged that other carbohydrates might be employed according to the invention.

Where legally permissible the present invention provides a method of treatment, prevention or amelioration of hangover or alcohol induced decline in well being, which method comprises administration of a formulation as described above. The invention also provides a method of suppression of appetite for alcohol, which method comprises administration of a formulation as described above.

1. A liquid preparation containing in a liquid volume of 250-300 ml the following constituents:
a) Maltodextrin preferably at 4 to 15 Dextrose Equivalents (DE), more preferably at 8 to 12 DE at 3 to 10% (w/v), even more preferably at 4 to 6% (w/v), and most preferred at 4.5% (w/v);
b) Maltodextrin preferably at 10 to 75 Dextrose Equivalents (DE), more preferably at 10 to 20 DE, most preferred 13 to 15 DE at 0.1 to 2% (w/v), but most preferred at 0.15% (w/v);
c) Maltodextrin preferably at 6 Dextrose Equivalents (DE) at 1 to 10% (w/v), but most preferred at 4% (w/v);
d) L-leucine at 0.025 to 5 grammes in total, more preferred 0.025 to 0.75 g in total and most preferred 0.05 g in total;
e) L-isoleucine at 0.001 to 5 grammes in total, more preferred 0.01 to 0.75 g in total and most preferred 0.025 g in total;
f) L-valine at 0.001 to 5 grammes in total, more preferred 0.01 to 0.75 g in total and most preferred 0.025 g in total;
g) Preferably L-ascorbic acid at about 0.1 g in total;
h) Preferably anhydrous citric acid at about 0.4 g;
i) Optionally sodium bicarbonate at about 0.3 g.

2. A preparation according to claim 1 containing in a liquid volume of 250-300 ml the following constituents:
a) about 6% (w/v) maltodextrins—made up of 50% MD6, 25% MD10 and 25% MD20;
b) about 25 mg each of valine and isoleucine;
c) about 50 mg of leucine;
d) about 60 mg of ascorbic acid; and
e) about 400 mg of citric acid.

3. A preparation according to claim 1 or 2 which is an aqueous solution of the constituents.

4. A preparation according to claim 1 or 2 in the form of an emulsion.

5. A preparation according to claim 1, 2, 3 or 4 additionally containing a sweetening/colouring/flavouring/preserving and/or carbonation agent.

6. A preparation according to claim 5 containing about 50 mg Aspartame.

7. A preparation according to any preceding claim wherein the maltodextrins are in part or completely substituted with longer or shorter polymeric maltodextrins.

8. A preparation according to any preceding claim wherein the maltodextrins have been substituted in part or completely with a different carbohydrate monomer, oligomer or polymer that does not provoke insulin secretion.

9. A preparation according to any preceding claim wherein the BCAAs are substituted in part or completely with their pharmacologically or physiologically active analogues.

10. A dry formulation which, when dissolved or suspended in about 250-300 ml of ligand, provides a preparation according to any preceding claim.

11. A dry formulation according to claim 10 in powder form.

12. A dry formulation according to claim 10 in particulate form.

13. A method for making a dry formulation according to claim 11 which comprises blending said dry constituents.

14. A method for making a dry formulation according to claim 12 which comprises mixing said dry constituents in particulate form.

15. Use of a preparation or formulation according to any one of claims 1 to 12 in a method for treatment/prevention/amelioration of alcohol induced hangover and/or alcohol induced decline in well being.

16. Use of a preparation or formulation according to any one of claims 1 to 12 in a method for suppression of alcoholic appetite.

17. A method of treatment/prevention/amelioration of alcohol induced hangover and/or alcohol induced decline in well being which comprises administration of a preparation according to any one of claims 1 to 9.

18. A method of suppression of alcoholic appetite which comprises administration of a preparation according to any one of claims 1 to 9.
